(19) United States
(12) Patent Application Publication
(10) Pub. No.: US 2006/0178561 A1
(43) Pub. Date: Aug. 10, 2006

(54) Endoscope apparatus

(75) Inventors: Sumito Nakano, Tokyo (JP); Kiyotomi Ogawa, Tokyo (JP); Mitsuo Obata, Tokyo (JP)

Correspondence Address: OSTROLENK FABER GERB & SOFFEN, 1180 AVENUE OF THE AMERICAS, NEW YORK, NY, 100368403, US

(73) Assignee: Olympus Corporation

(21) Appl. No.: 11/346,786

(22) Filed: Feb. 3, 2006

(30) Foreign Application Priority Data
Feb. 7, 2005 (JP) 2005-031126

(51) Int. Cl. A61B 1/04

(52) U.S. Cl. 600/117; 600/118; 600/109

(57) ABSTRACT

A measuring endoscope apparatus captures a target of measurement, generates an original image, and performs a measurement based on the position of the measurement point on the original image. The apparatus can easily specify the measurement point with high precision, and realize high precision measurement. For example, the measurement point by a re-sampling image generated by moving sampling points by a spacing smaller than a pixel spacing of the original image obtained by capturing a target of measurement.

### CROSS REFERENCE TO RELATED APPLICATION

This application claims benefit of Japanese Application No. 2005-031126, filed Feb. 7, 2005, the contents of which are incorporated by this reference.

### BACKGROUND OF THE INVENTION

1. Field of the Invention

The present invention relates to a measuring endoscope apparatus for capturing a target of measurement, generating an original image, and performing a measurement based on the position of the measurement point on the original image.

2. Description of the Related Art

Recently, a measuring endoscope apparatus is used in measuring the scratch and loss of various machine parts. The measuring endoscope apparatus captures a target of measurement, generates an original image, and performs a measurement based on the position of the measurement point on the read original image.

Japanese Published Patent Application No. H4-332523 proposes a method for enlarging an image and specifying a measurement point on the enlarged image as a technique of specifying a measurement point on an original image. In this method, a pixel corresponding to a measurement point is selected and specified from among the pixels on the enlarged image, and a measurement is made based on the position of the original image corresponding to the specified pixel. Furthermore, the position of the specified measurement point on the original image can be calculated in a unit of a reciprocal of a magnification. Therefore, based on the calculated position, a unit finer than pixel spacing of an original image can be measured based on the calculated position.

Described below is an example of specification of a measurement point in the conventional method. For example, FIG. 1 shows an original image of a target of measurement. As shown in FIG. 1, the background of the original image is white, and each of the two black lines is two pixels wide and the lines form a right angle. The measurement point is an enlarged area obtained by enlarging the area including the center of the intersection point of the two lines.

FIG. 2 shows an enlarged image of the enlarged area. On the enlarged image shown in FIG. 2, a “+” mark indicating a specified point is displayed. In the above-mentioned technology, a measurement point is specified on the enlarged image shown in FIG. 2, and an arithmetic operation is performed based on the position of the pixel in the original image corresponding to the pixel in the specified enlarged image. As described above, the position on the original image corresponding to the pixels on the specified enlarged image is calculated in a unit of the reciprocal of the magnification, thereby possibly performing a measurement based on the position.

### SUMMARY OF THE INVENTION

The measuring endoscope apparatus according to an aspect of the present invention having an original image acquisition unit for acquiring an image by sampling a captured target in a pixel unit as an original image, and a re-sampling image generation unit for generating an image by re-sampling the original image at desired position on all or a part of the area of the original image includes: a sampling point travel unit for moving sampling points corresponding to the pixels in all or a part of area of the original image in a unit finer than pixel spacing of the original image in the re-sampling image generation unit; a measurement point position specification unit for specifying the position of the measurement point on the original image in the unit finer than the pixel spacing of the original image by moving the sampling points to a desired position by the sampling point travel unit; and a measurement unit for performing a measurement based on the position of the specified measurement point in a unit finer than the pixel spacing of the original image.

With the above-mentioned configuration, when the position of a measurement point is specified in a unit finer than the pixel spacing of an original image, a position having a necessary feature can be easily determined. Additionally, since the unit of position specification of a measurement point can be arbitrarily set, high precision measurement can be performed. Furthermore, a measurement point can be easily specified by enlargement by arbitrary magnification.

### BRIEF DESCRIPTION OF THE DRAWINGS

FIG. 1 shows an example of an original image to be measured;

FIG. 2 shows a simple enlarged image in the enlarged area shown in FIG. 1;

FIG. 3 is an explanatory view of a measuring endoscope apparatus according to an embodiment of the present invention;

FIG. 4 is a block diagram of the configuration of the measuring endoscope apparatus;

FIG. 5 is an explanatory view of a remote controller;

FIG. 6 is a perspective view of the configuration in which a direct-view stereo optical adapter is attached to the end portion of a measuring endoscope;

FIG. 7 is a sectional view along A-A shown in FIG. 6;

FIG. 8 shows the method of obtaining the 3-dimensional coordinates of the measurement point by the stereometry;

FIG. 9A is a flowchart showing the flow of performing a measurement by the measuring endoscope apparatus;

FIG. 9B is a flowchart explaining the specification of a measurement point;

FIG. 9C is a flowchart explaining the setting of an enlarged area;

FIG. 9D is a flowchart explaining an enlarged image generating process;

FIG. 9E is a flowchart explaining the travel of a sampling point;

FIG. 10A shows two read right and left original images;

FIG. 10B shows the measurement screen when an enlarged image is displayed by pointing to the vicinity of the measurement point;

FIG. 10C shows the measurement screen including the enlarged image when the sampling point is moved;

FIG. 10D shows the measurement screen when the magnification is changed to six times;

FIG. 10E shows the measurement screen when the unit of the amount of travel of a sampling point as the pixel spacing of the original image, and the magnification is changed to six times;

FIG. 10F shows the measurement screen;

FIG. 10G shows the measurement screen including the measurement result when the difference between two points are measured;

FIG. 11 shows the enlarged image of a sampling point travel image generated by linear interpolation;

FIG. 12A shows the original image of horizontal 6 pixels×vertical 1 pixel;

FIG. 12B shows the brilliance at the sampling point of the original image;

FIG. 12C shows the brilliance of a sampling point travel image;

FIG. 12D shows the brilliance at the sampling point of the original image when the number of pixels is increased for enlargement;

FIG. 12E shows the generation of an enlarged image from the brightness information shown in FIG. 12D;

FIG. 13 shows the sampling point of the original image and a moved sampling point.

FIG. 14A shows the case where an enlarged image shows vertical stripe noise;

FIG. 14B shows an example of a brilliance signal in this case;

FIG. 14C shows an example of reducing noise when a filter is applied; and

FIG. 14D shows an example of the brilliance signal in this case.

### DESCRIPTION OF THE PREFERRED EMBODIMENTS

The embodiments of the present invention are explained by referring to the attached drawings.

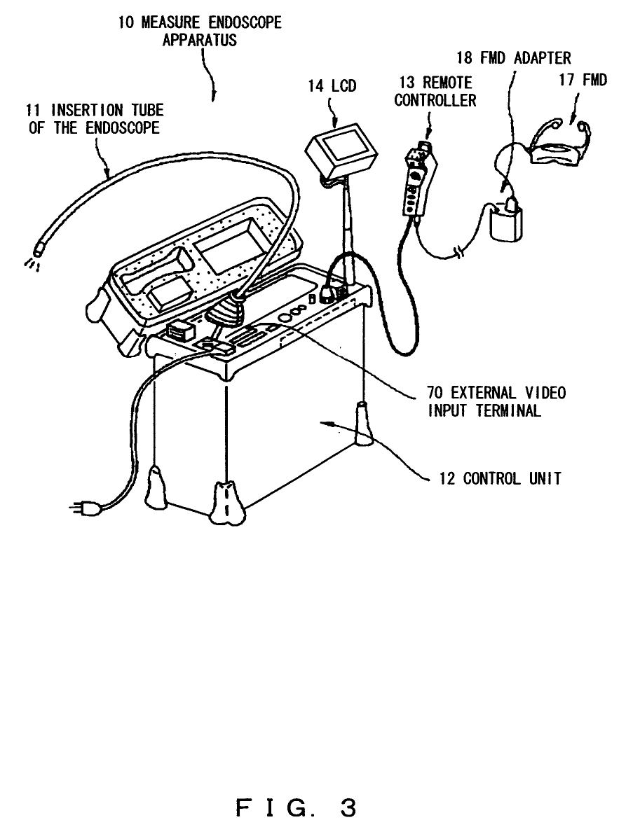

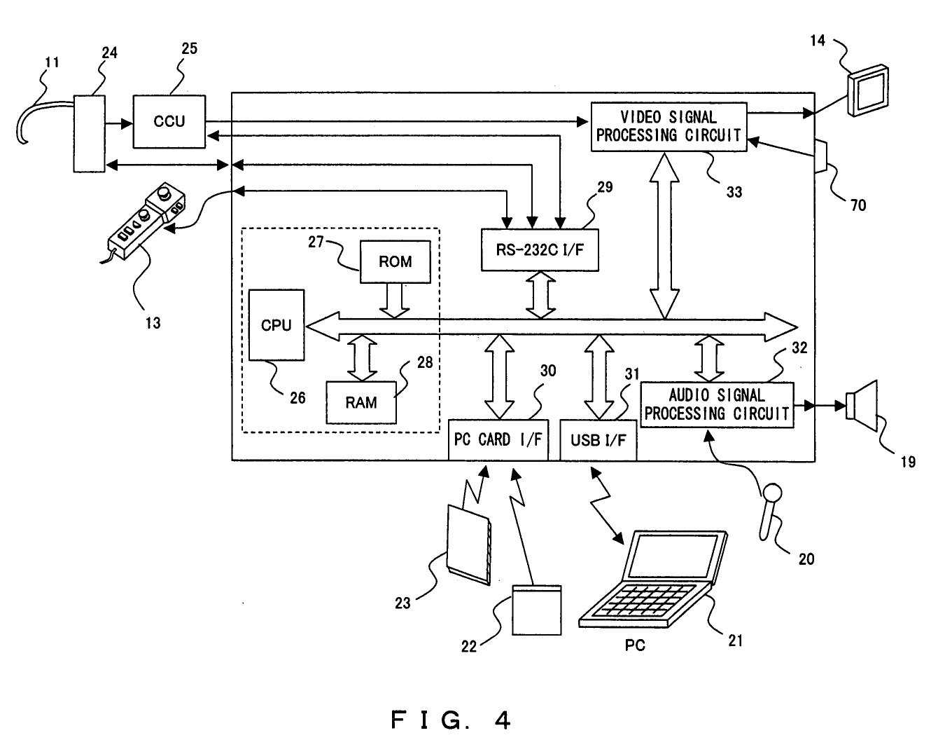

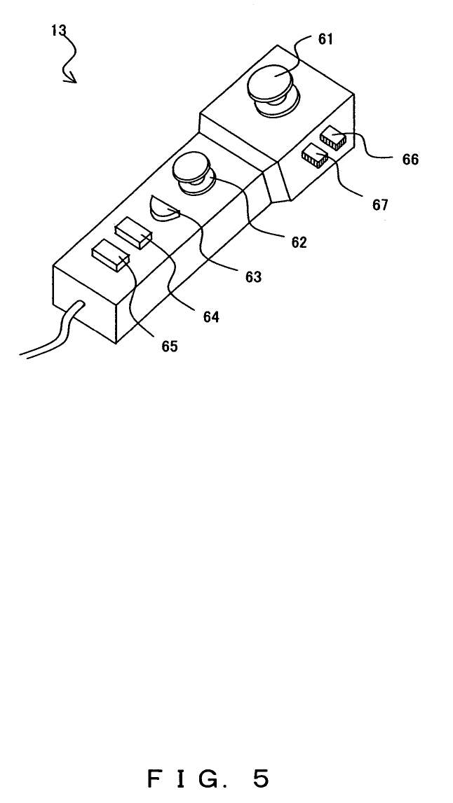

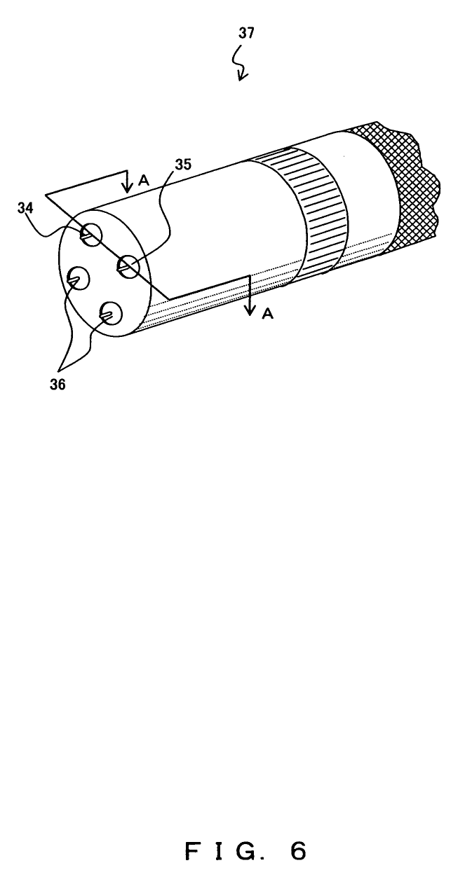

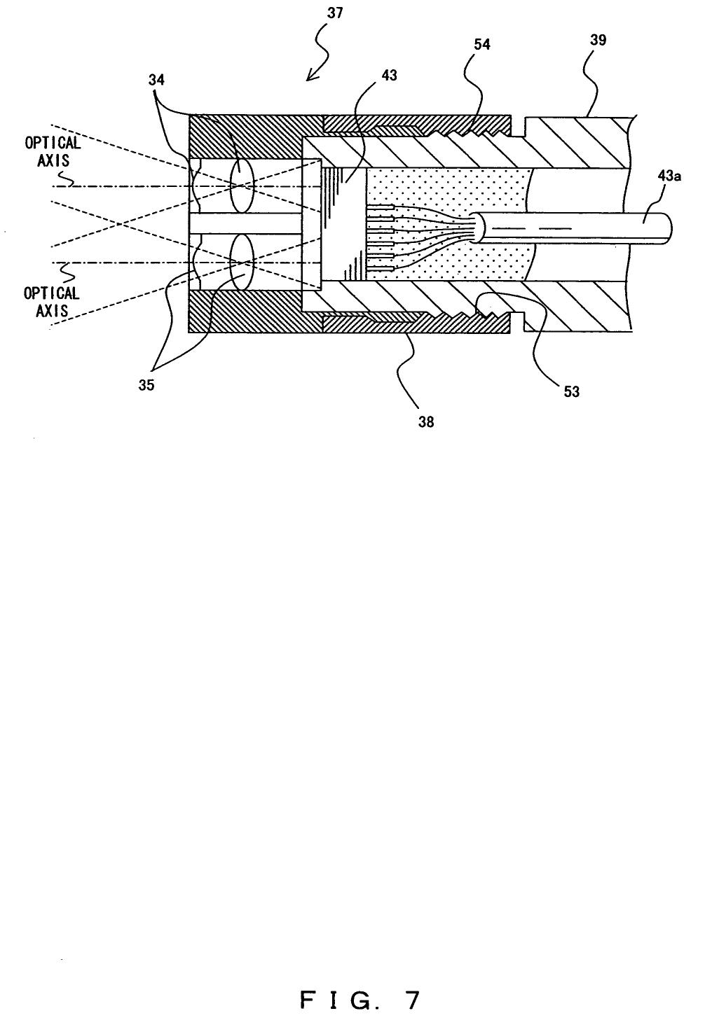

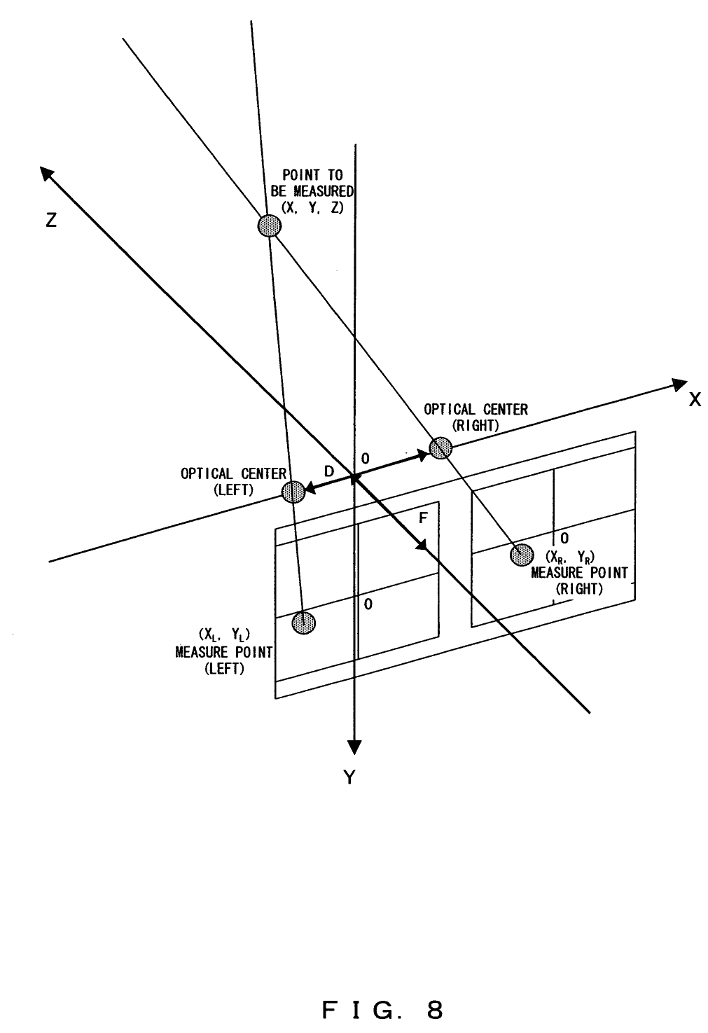

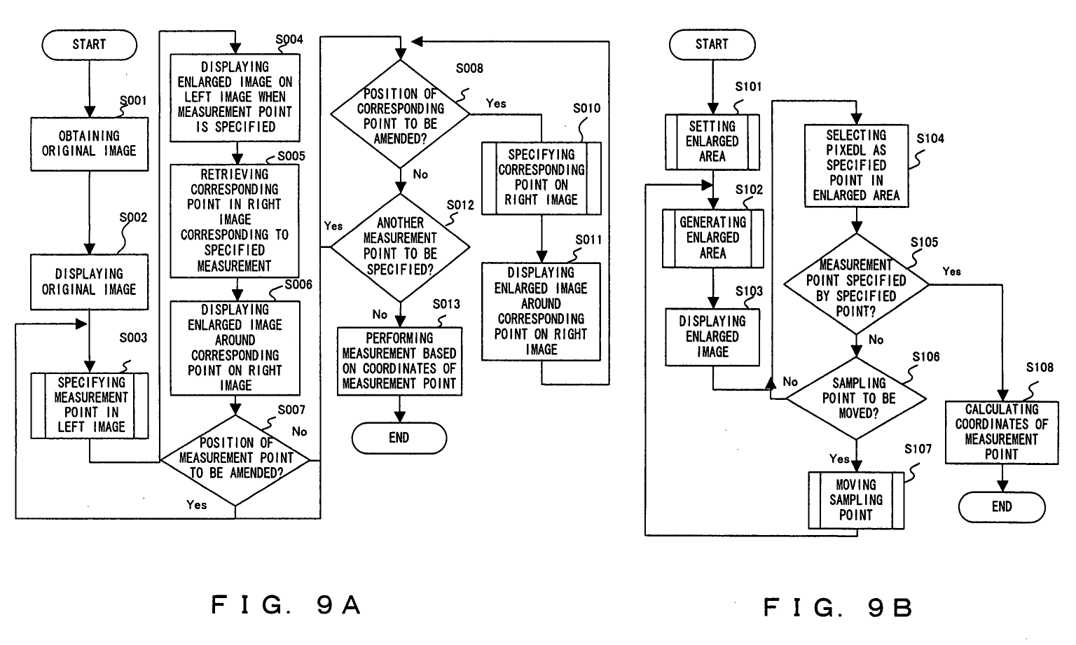

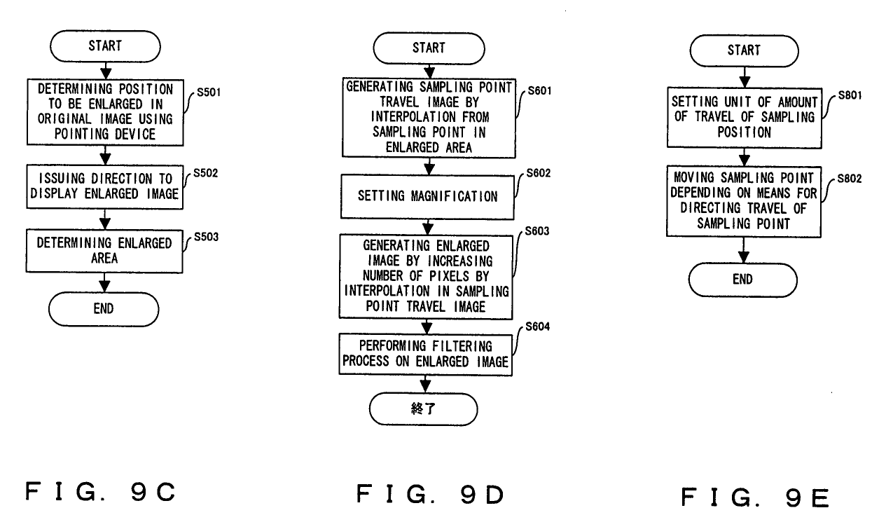

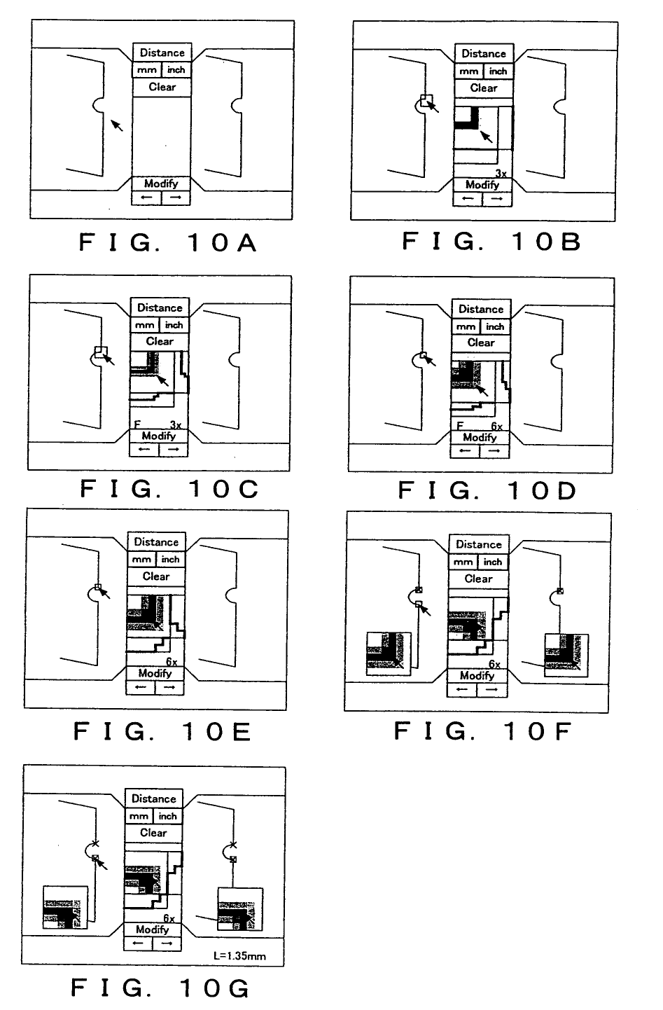

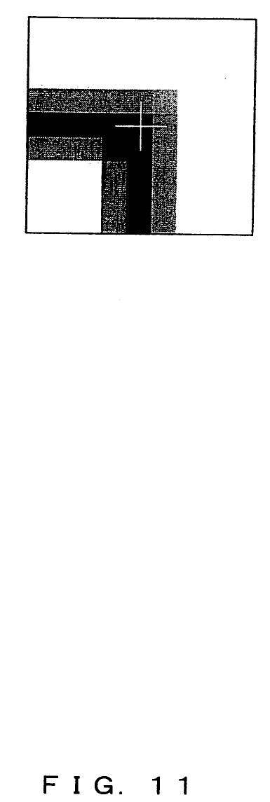

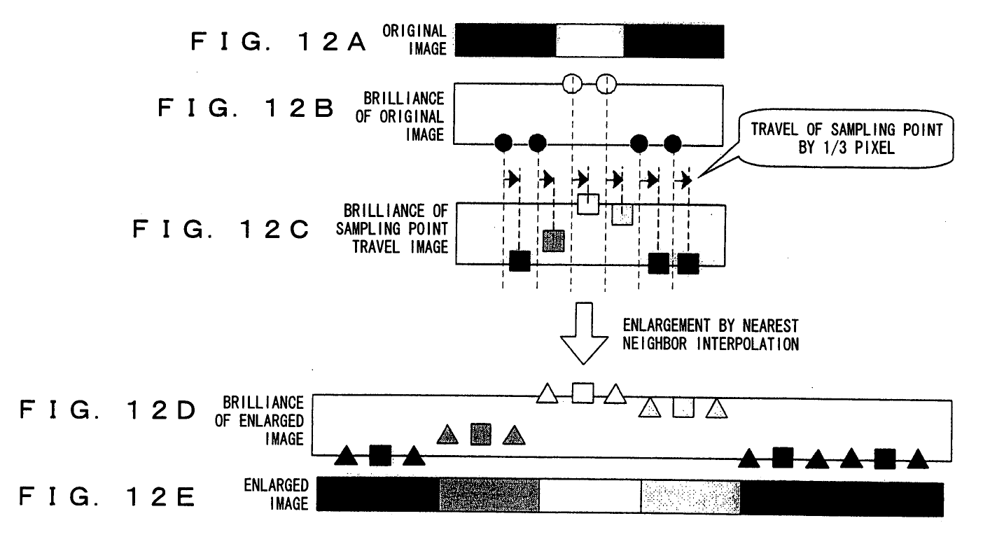

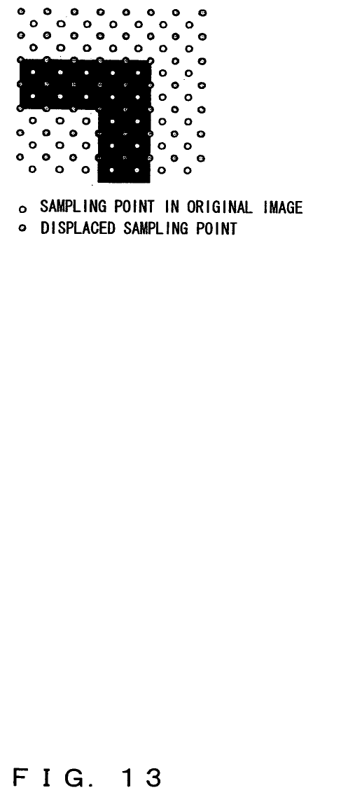

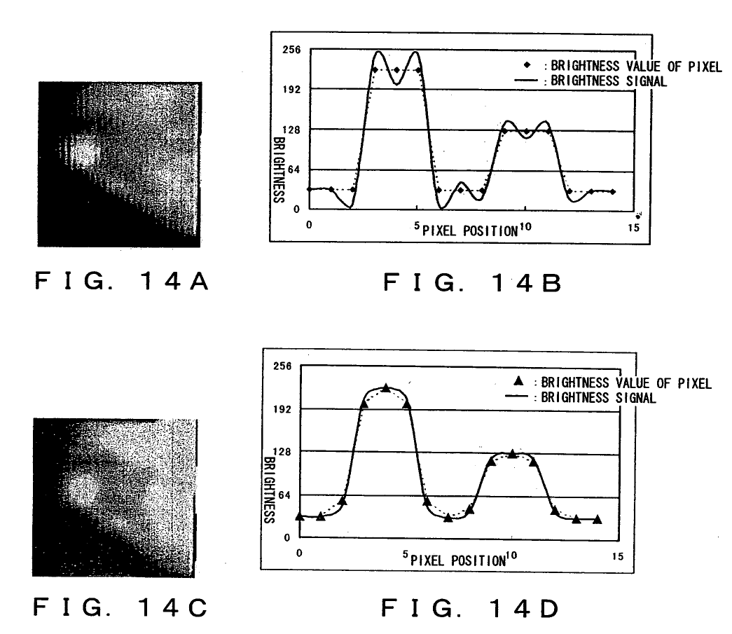

FIGS. 3 through 14 relate to the embodiments of the present invention. FIG. 3 is an explanatory view of a measuring endoscope apparatus according to an embodiment of the present invention. FIG. 4 is a block diagram of the configuration of the measuring endoscope apparatus. FIG. 5 is an explanatory view of a remote controller. FIG. 6 is a perspective view of the configuration in which a direct-view stereo optical adapter is attached to the end portion of a measuring endoscope. FIG. 7 is a sectional view along A-A shown in FIG. 6. FIG. 8 shows the method of obtaining the 3-dimensional coordinates of the measurement point by the stereometry. FIG. 9 is a flowchart showing the flow of performing a measurement by the measuring endoscope apparatus. FIG. 10 is an explanatory view of the stereometry execution screen. FIG. 11 shows an enlarged image of a sampling point travel image. FIG. 12 shows a sampling point travel image and an explanatory view showing the principle of generating an enlarged image. FIG. 13 shows a sampling point of an original image and a moved sampling point. FIG. 14 shows reduced noise when a filter is applied.

First, a measuring endoscope apparatus 10 comprises: an insertion tube 11 of the endoscope configured such that an optical adapter including the function of performing stereometry as shown in FIG. 3 can be designed to be freely attached and removed; a control unit 12 storing the insertion tube 11 of the endoscope; a remote controller 13 for performing a necessary operation to control various operations of the entire system of the measuring endoscope apparatus 10; a liquid crystal monitor (hereinafter referred to as an LCD) 14 for displaying an endoscope and operation control contents (for example, a process menu), etc.; a face mount display (hereinafter referred to as an FMD) 17 capable of three-dimensionally displaying a normal endoscopic image or the endoscopic image as a pseudo stereo image; and an FMD adapter 18 for providing image data for the FMD 17.

The configuration of the system of the measuring endoscope apparatus 10 is explained in detail by referring to FIG. 4. As shown in FIG. 4, the insertion tube 11 of the endoscope is connected to an endoscope unit 24. The endoscope unit 24 is loaded into the control unit 12 shown in FIG. 3. The endoscope unit 24 is configured to comprise a light source device for obtaining illuminating light necessary during capturing and a motor-driven bending device for electrically and freely bending the insertion tube 11 of the endoscope. A capture signal from a solid-state image pickup device 43 (refer to FIG. 7) at the tip of the insertion tube 11 of the endoscope is input to a camera control unit (hereinafter referred to as a CCU) 25. The CCU 25 transforms a provided capture signal to a video signal such as an NTSC signal, etc., and provides it for a central processing circuit group in the control unit 12.

The central circuit group loaded into the control unit 12 comprises a CPU 26 for controlling such that various functions can be executed and operated based on the main program as shown in FIG. 4, ROM 27, RAM 28, a PC card interface (hereinafter referred to as a PC card I/F) 30, a USB interface (hereinafter referred to as a USB I/F) 31, an RS-232C interface (hereinafter referred to as an RS-232C I/F) 29, an audio signal processing circuit 32, and a video signal processing circuit 33. The CPU 26 executes a program stored in the ROM 27, and controls the operations of the entire system by controlling various circuit units so that processes can be performed depending on the purpose.

The RS-232C I/F 29 is connected to the CCU 25, the endoscope unit 24, and the remote controller 13. The remote controller 13 controls and operates the CCU 25 and the endoscope unit 24. The RS-232C I/F 29 is designed to perform necessary communications to control the operation of the CCU 25 and the endoscope unit 24 based on the operation by the remote controller 13.

The USB I/F 31 is an interface for electrical connection between the control unit 12 and a personal computer 21. When the control unit 12 is connected to the personal computer 21 through the USB I/F 31, the personal computer 21 can also control various operations such as issuing an instruction to display an endoscopic image, image processing during measurement in the control unit 12, and can input/output necessary control information, data, etc. for various processes with the personal computer 21.

The PC card I/F 30 is designed such that a PCMCIA memory card 23 and a Compact Flash (R) memory card 22 can be freely connected. That is, when any of the memory cards is inserted, the control unit 12 regenerates data such as the control processing information, image information, etc. stored in the memory card as a recording medium by the control of the CPU 26, fetches the data in the control unit 12 through the PC card I/F 30, or provides the data such as control processing information, image information, etc. for the memory card through the PC card I/F 30, and stores them.

The video signal processing circuit 33 combines the video signal from the CCU 25 with the display signal based on the operation menu generated by the control of the CPU 26 so that a composite image of the endoscopic image provided from the CCU 25 and the operation menu of graphics can be displayed, performs a necessary process to display the composite image on the screen of the LCD 14, and provides the result for the LCD 14. Thus, the LCD 14 displays the composite image of the endoscopic image and the operation menu. The video signal processing circuit 33 can also perform the process of displaying a simple image such as an endoscopic image, an operation menu, etc.

The control unit 12 shown in FIG. 3 is separately provided with an external video input terminal 70 for inputting a video to the video signal processing circuit 33 without using the CCU 25. When a video signal is input to the external video input terminal 70, the video signal processing circuit 33 outputs the composite image before the endoscopic image from the CCU 25 on a priority basis.

The audio signal processing process 32 provides an audio signal collected by a microphone 20 and generated, and stored in a recording medium such as a memory card, etc., an audio signal obtained by regeneration by a recording medium such as a memory card, etc., or an audio signal generated by the CPU 26. The audio signal processing circuit 32 performs a necessary process (amplifying process, etc.) for regeneration on the provided audio signal, and outputs it to a speaker 19. Thus, the speaker 19 regenerates the audio signal.

The remote controller 13 comprises a joystick 61, a lever switch 62, a freeze switch 63, a store switch 64, a measurement execution switch 65, a WIDE switch 66 for enlarged display switch, and a TELE switch 67 as shown in FIG. 5.

In the remote controller 13, the joystick 61 performs a bending operation on the tip of the endoscope, freely provides an operation instruction at any angle. For example, the switch can be pressed down, and an instruction for a fine adjustment to a bending operation can be issued. The lever switch 62 is used in determining an option by moving the pointer and pressing it down when various menu operations and measurements are performed, and is designed to have substantially the same form as the joystick 61. The freeze switch 63 is used in displaying an image on the LCD 14. The store switch 64 is used when a static image is displayed by pressing the freeze switch 63 and the static image is recorded on the PCMCIA memory card 23 (FIG. 4). The measurement execution switch 65 is used when measurement software is executed. The WIDE switch 66 for enlarged display switch and the TELE switch 67 are used when an endoscopic image is enlarged or reduced. The freeze switch 63, the store switch 64, and the measurement execution switch 65 are designed as, for example, on/off press-button.

An endoscopic image captured by the insertion tube 11 of the endoscope is enlarged or reduced as necessary by the video signal processing circuit 33, and output to the LCD 14 or the external video input terminal 70. The control of the magnification for enlargement or reduction is performed by the WIDE switch 66 for enlarged display switch and the TELE switch 67. The control of the magnification when an enlarged image is displayed during measurement is also performed by the WIDE switch 66 for enlarged display switch and the TELE switch 67.

The control of enlargement and reduction of an endoscopic image captured by the insertion tube 11 of the endoscope and the control of the magnification when an enlarged image is displayed during measurement are performed by the configuration of two switches of the WIDE switch 66 and the TELE switch 67. However, there can be a case where it is hard or impossible to provide the two switches for the operation directive device such as a remote controller, etc. In this case, the control of enlargement and reduction can be performed by one switch. That is, each time the switch is pressed, the magnification can be increased or decreased to a predetermined magnification A, and after the predetermined magnification A is set, the magnification can be reduced or increased to a predetermined magnification B each time the switch is pressed. By repeating the control, the control for enlargement and reduction can be performed by one switch.

Next, the configuration of a stereo optical adapter as a type of optical adapter used for the measuring endoscope apparatus 10 according to the present embodiment is explained below by referring to FIGS. 6 and 7.

FIGS. 6 and 7 show the status of a stereo optical adapter 37 attached to an endoscope end portion 39. The stereo optical adapter 37 is designed to be fixed by a female screw 53 of a fixing ring 38 to be engaged with a male screw 54 of the 39.

A pair of illumination windows 36 and two objective lenses 34 and 35 are provided at the tip of the stereo optical adapter 37. The two objective lenses 34 and 35 form two images on the image pickup device 43 arranged in the endoscope end portion 39. Then, a capture signal obtained by the image pickup device 43 is provided for a signal line 43a and the CCU 25 through the endoscope unit 24 shown in FIG. 4, and after being transformed by the CCU 25 to a video signal, it is provided for the video signal processing circuit 33. The video signal includes a brilliance value, or a brilliance value and a chrominance difference value. An image generated by the capture signal provided for the CCU 25 is referred to as an original image.

The method for obtaining 3-dimensional coordinates of a measurement point by the stereometry is explained below by referring to FIG. 8. The coordinates of the measurement point of the original image captured by left and right optical systems are respectively (XL, YL) and (XR, YR), and the 3-dimensional coordinates of the measurement point is (X, Y, Z), while the origins of (XL, YL) and (XR, YR) are respectively the intersection points of the optical axis of the left and right optical centers and the image pickup device 43, and the origin of the (X, Y, Z) is the intersection point of the left and right optical systems. If the distance between the left and right optical centers is D, and the focal length is F, then the following equations hold in the triangulation method.
X=t×XR+D/2
Y=t×YR
Z=t×F
where t=D/(XL−XR)

Thus, when the coordinates of the measurement point of an original image are determined, the 3-dimensional coordinates of the measurement point are determined using the known parameters D and F. By obtaining some 3-dimensional coordinates, a measurement can be performed on various targets such as the distance between the two points, the distance between the line connecting the two points and one point, an area, a depth, the shape of a surface, etc.

Relating to the measuring endoscope apparatus with the above-mentioned configuration, the processing operation according to the present embodiment is explained below by referring to FIGS. 9 through 13. FIG. 9 is a flowchart of the stereometry. FIG. 10 shows the screen of the stereometry. The image shown in FIG. 10 shows an example in which there is chipping detected in the turbine blade as an engine part of an aircraft, and the measurement screen of the case where the outermost width of the chipping is measured.

First, when the measurement execution switch 65 provided for the joystick 61 is pressed, the image generated by sampling in a pixel unit is obtained as an original image in step S001 in the measurement flow shown in FIG. 9A, and displayed on the display device in step S002. FIG. 10A shows a measurement screen formed by the left and right original images, icons in dicating the measuring operation, and a pointer specifying the position by the lever switch 62.

Then, a measurement point is specified in the left image in step S003. The specification of the measurement point is performed in the measurement point specification flow shown in FIG. 9B. First, in step S101, an enlarged area as a portion to be enlarged in the original image is set. The setting of the enlarged area is performed according to the enlarged area setting flow shown in FIG. 9C. That is, if the lever switch 62 is operated and the position near the measurement point of the original image is specified in step S501, and an enlarged image display instruction is issued in step S502, then an enlarged area is determined in step S503. In the present embodiment, the enlarged area is an area of a predetermined range with the position specified by the lever switch 62 defined as the center.

Then, in step S102, an enlarged image is generated. The generation of the enlarged image is performed according to the flow shown in FIG. 9D. First, in step S601, an image is generated based on the position of the sampling point in the enlarged area. The position of the sampling point in the enlarged area is the position of the sampling operation when the original image is first acquired, and is moved in step S107 described later.

When the position of the sampling point of the enlarged area is moved, it is displaced from the position of the sampling operation when the original image is acquired. Therefore, an image is generated by interpolation from the pixel in the original image. The interpolating method is executed by the nearest neighbor interpolation, the linear interpolation, the bicubic interpolation, etc. The image generated in step S601 is a sampling point travel image.

Then, in step S602, a magnification is set from the number of presses of the WIDE switch 66 for enlarged display switch or the TELE switch 67, and an enlarged image is generated by increasing the number of pixels of the sampling point travel image by the amount corresponding to the magnification by an interpolating operation in step S603. The interpolating method is executed by the nearest neighbor interpolation, the linear interpolation, the bicubic interpolation, etc. In step S604, the filtering process described later is performed on the enlarged image.

In step S103, the size and the position of the enlarged image are determined and displayed in step S103. The display position can be superposed on the original image. In this case, the display position of the enlarged image is set at a predetermined distance from the enlarged area of the original image, thereby preventing the enlarged area and the vicinity from being lost on the display.

In step S104, a pixel as a specified point is selected in the enlarged image. Then, a cursor indicating the specified point is displayed on the selected pixel. The pixel as a specified point can be at a predetermined fixed position in the enlarged image. On the selected pixel, a cursor indicating a specified point is displayed. The pixel as a specified point can be a predetermined fixed position in the enlarged image.

FIG. 10B shows the measurement screen when the vicinity of the measurement point is pointed to, and an enlarged image is displayed. On the measurement screen, the enlarged image and the cursor indicating the specified point are displayed at the center of the screen. A graph indicating the brilliance of the pixel in the vertical and horizontal directions from the specified point is displayed on the right and below the enlarged image, and the brilliance of the specified point and the vicinity can be confirmed. Additionally, “3×” indicating the magnification of three times is displayed.

In step S105, it is determined whether or not a measurement point has been specified by the specified point. If the measurement point has not been specified, control is passed to step S106. If the measurement point has been specified, the lever switch 62 is pressed, and control is passed to step S108.

In step S106, it is determined whether or not the sampling point is moved. If there is a measurement point in the enlarged image, and if it is not necessary to move the sampling point because the sampling point matches the measurement point, then control is passed to step S104, and the displayed measurement point is selected as a specified point. When there is a measurement point in the enlarged image but the sampling point does not match the measurement point, and it is necessary to move the sampling point, or when there is no measurement point in the enlarged image, control is passed to step S107. In step S107, the sampling point is moved so that the measurement point can be specified by the specified point in the enlarged image. The travel of the sampling point is performed according to the flow shown in FIG. 9. First, to quickly move the specified point to the position near the measurement point, the unit of the amount of travel of the sampling point is set to the unit of the pixel spacing of the original image in step S801.

Next, in step S802, the lever switch 62, the specified point is moved toward the measurement point. To attain this, the joystick 61 is pressed, and the unit of the amount of travel of the sampling point is switched to the unit finer than the pixel spacing to specify the measurement point with high precision. Then, the position of the sampling point is moved by the lever switch 62, and the specified point is moved to the measurement point. When the unit of the amount of travel of the sampling point is set finer than the pixel spacing, the icon “F” is displayed (refer to FIG. 10C explained later).

By performing the process, the sampling point is quickly moved toward the measurement point with the amount of travel used as pixel spacing when the specified point is apart from the measurement point. Then, the unit of the amount of travel is set in a unit finer than the pixel spacing, thereby correctly moving the specified point toward the measurement point. Therefore, in the process of the present example, the user can easily set the measurement point.

The travel of the sampling point can be performed in the following procedure. First, in step S801, the unit of the amount of travel of the sampling point is set by a press of the joystick 61 or the freeze switch 63. Next, according to the setting in step S801, the specified point is moved to the measurement point by the lever switch 62 in step S802.

Then, in step S107, when the position of the sampling point is moved, the enlarged area is moved correspondingly. After the travel of the sampling point, control is passed to step S102, the enlarged image is generated again. FIG. 10C shows the measurement screen including the enlarged image when the sampling point is moved. FIG. 10D shows the measurement screen when the magnification is changed to six times. FIG. 10E shows the measurement screen when the unit of the amount of travel of the sampling point is set as the pixel spacing of the original image, and the sampling point is moved from the status shown in FIG. 10D.

Thus, by switching the unit of the amount of travel of the sampling point, rough and precise travel can be performed, and a measurement point can be specified in a short time.

Then, after the specified point travels to the measurement point in the above-mentioned process, the sampling point is determined by pressing the lever switch 62 in step S105, and the position of the measurement point in the original image is calculated from the position of the specified point in step S108.

In step S004, the enlarged image at the time when specification is performed in step S003 is superposed on the left image and displayed. In step S005, the corresponding point in the right image corresponding to the measurement point specified in step S003 is searched. The search is performed in the unit finer than the pixel spacing of the original image in the template matching method on the existing image.

In step S006, the vicinity of the corresponding point in the right image is enlarged as in the enlargement of the left image, and superposed on the right image and displayed.

FIG. 10F shows the measurement screen at this time. By displaying the enlarged image of the original image, the previous measurement point and the matching result of the corresponding point of the right image can be correctly confirmed during the specification of the next measurement point, thereby correctly preventing error in measurement.

Then in step S007, it is determined whether or not the position of the measurement point on the left screen is to be amended. If the position of the measurement point on the left screen is to be amended, the lever switch 62 is operated, the icon “←” on the measurement screen is selected, control is returned to step S003, and the measurement point is specified again. On the other hand, if it is not to be amended, control is passed to step S008.

In step S008, it is determined whether or not the position of the corresponding point on the right screen is to be amended. If it is to be amended, the lever switch 62 is operated, the icon “→” on the measurement screen is selected, control is passed to step S010, and the corresponding point is specified in the right image as in the specification of the measurement point in the left image. Then, in step S011, the vicinity of the corresponding point in the right image is display as in the process in step S006.

In the determination in step S007 and S008, the enlarged images of the vicinities of the measurement point of the left image and the corresponding point of the right image are largely displayed respectively on the left and right screens to confirm whether or not the measurement point and the corresponding point have been correctly specified.

In step S008, when the position is not amended, control is passed to step S012, and it is determined whether or not another measurement point is specified. When it is specified, control is returned to step S003. If it is not specified, control is passed to step S013. In this process, a measurement is performed based on the position of the measurement point specified as described above. FIG. 10G shows the measurement screen including the measurement result when the distance between two points is measured.

In the example of the measurement result shown in FIG. 10G, the measurement unit is “mm”, but the measurement unit can be switched between “mm” and “inch” on the screen. When the measurement units are switched, the display of the measurement result is also changed to the set unit. Thus, the measurement units can be switched with optional timing while continuing the measuring operation. This works when the unit generally used is different from the unit written in the checking manual, and when the setting is wrong. The measurement unit can also be changed by the setting of the menu.

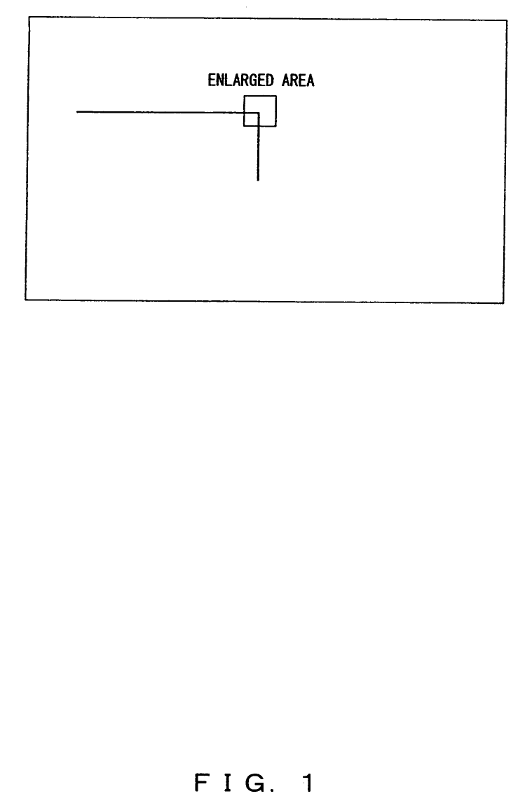

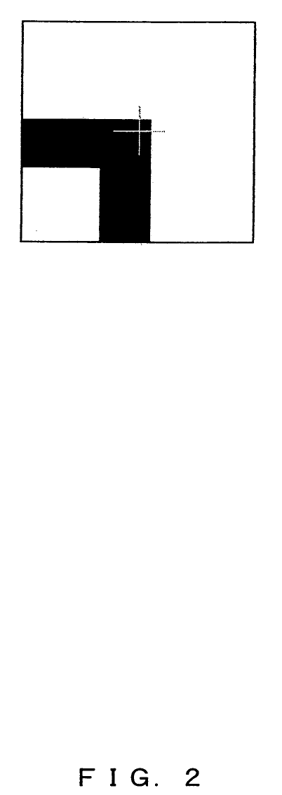

The details of the specification of a measurement point are explained below by referring to an example of an original image shown in FIG. 1. First, the enlarged area shown in FIG. 1 is enlarged as shown in FIG. 2. The number of pixels for enlargement is increased in the nearest neighbor interpolation, the unit of the amount of travel of the sampling point is set to 0.1 pixel so that the specified point can be moved to the center of the intersection point of two lines, and the sampling point is 0.5 pixel moved to the left and 0.5 pixel moved down. In this process, a pseudo sampling image is generated in the linear interpolation, and the enlarged image of the image is generated. FIG. 11 shows the enlarged image at this time.

The principle of generating a pseudo sampling point travel image and its enlarged image is explained below by referring to FIG. 12. The original image is formed by 6 horizontal pixels×1 vertical pixel as shown in FIG. 12A. The central two pixels are white, and the other surrounding pixels are black. FIG. 12B shows the brilliance of the sampling point of the original image.

When the sampling point is moved ⅓ pixel to the right of the original image, the brilliance of the moved sampling point is calculated by the interpolation from the pixel of the original image, and the brilliance of the sampling point travel image is shown in FIG. 12C. If the number of pixels of the sampling point travel image is increased for enlargement, the brilliance is changed as shown in FIG. 12D. From the brilliance, the enlarged image shown in FIG. 12E is generated.

The principle of generating the enlarged image shown in FIG. 11 is described below. FIG. 13 shows the sampling point in the original image and the moved sampling point. In the original image shown in FIG. 2, the black line spans two sampling points. For example, in the enlarged image, the line having the thickness of 2 in the original image is a simply enlarged image. On the other hand, when the sampling point is moved, the position spanning the two pixels in the black line is black, and the boundary position between white and black is gray by interpolation. Therefore, in the enlarged image, the position of the specified point is black, but the surrounding portion is gray.

As described above, in the enlarged image of a pseudo sampling image, the color of the position of a specified point is definite, but the vicinity is displayed in the color of every second pixel in the original image from the specified point. Therefore, it is easy to discriminate the color of the specified point from the colors of the other points As a result, a desired point can be easily specified in a unit finer than the pixel spacing of the original image.

There can be vertical stripe noise shown in FIG. 14A occurring in the enlarged image output to the LCD 14. FIG. 14B shows an example (solid line) of a brilliance signal output to the display device when an original image has the brilliance shown by the dotted line. Conventionally, a filtering process is performed on the entire picture output to the LCD 14, and the noise on the enlarged image is reduced or removed. However, since a filter is applied to the entire picture, the picture becomes blurred. With the endoscope apparatus according to the present invention, the noise occurring in the enlarged image can be reduced or removed by performing the filtering process only on the generated enlarged image. As a filter, an arithmetic operation for reducing the ratio of a change of the signal of an enlarged image as described below can be applied. (A) A filter for defining the brilliance of each pixel as a weighted average with the right pixel, that is,
L_{A}(x, y)=p×L_{B}(x, y)+q×L_{B}(x+1, y)
where p+q=1

For example, p=q=1/2 (arithmetic mean) (B) A filter for defining the brilliance of each pixel as a weighted average with the right and left pixels, that is,
L_{A}(x, y)=p×L_{B}(x−1, y)+q×L_{B}(x, y)+r×L_{B}(x+1, y)
where p+q+r=1

For example, p=r=1/4, q=1/2 (weighted average)

Otherwise, p=r=0.274, q=0.452 (a normalized Gaussian filter using a Gaussian function f (x)=exp (−xˆ2/2 σ), σ=1)

where L_{B} (x, y) indicates the brilliance value of the image before the filtering process, L_{A} (x, y) indicates the brilliance value of the image after the filtering process, and (x, y) indicates the position of the pixel in the image.

FIG. 14C shows an enlarged image when the filter (B) (p=r=1/4, r=1/2) is applied, and the vertical stripe noise is reduced. FIG. 14D shows an example in which a filter (B) is applied to the original image shown in FIG. 14B. The dotted line and the solid line shown in FIG. 14D respectively show the brilliance of an enlarged image to which a filter is applied and the brilliance signal output to the display device.

Therefore, according to the present embodiment, the unit of the amount of travel of a sampling point can be arbitrarily set more minutely, and the measurement point can be specified with high precision unlike the conventional method dependent of the magnification. In the process of the travel of the sampling point, a change of an enlarged image can be easily checked, and the image can be moved to a desired measurement point.

Furthermore, by selecting an appropriate interpolation algorithm, the visibility of an enlarged image can be improved, and the measurement point can be easily specified. Additionally, the magnification can be specified not only by an integer, but also by a real number, thereby displaying an image by a desired magnification.

In the explanation of the embodiments above, the loss of a turbine blade as engine parts of an aircraft is explained, but the measuring endoscope apparatus according to the present invention can also be used in measuring a scratch, a loss, etc. of various equipment parts.

According to the present invention, when the position of a measurement point is specified in a unit finer than the pixel spacing of an original image, a point having a necessary feature can be easily determined.

Furthermore, since the unit of the specification of the position of a measurement point can be arbitrarily set, the measurement can be performed with higher precision than in the conventional method.

In addition, a measurement point can be more easily specified by enlargement by an arbitrary magnification.

What is claimed is:

1. A measuring endoscope apparatus having an original image acquisition unit for acquiring an image by sampling a captured target in a pixel unit as an original image, and a re-sampling image generation unit for generating an image by re-sampling the original image at a desired position on all or a part of the area of the original image, comprising:
a sampling point travel unit moving sampling points corresponding to the pixels in all or a part of area of the original image in a unit finer than pixel spacing of the original image in the re-sampling image generation unit;
a measurement point position specification unit specifying a position of a measurement point on an original image in the unit finer than the pixel spacing of the original image by moving the sampling points to a desired position by the sampling point travel unit; and
a measurement unit performing a measurement based on the position of the specified measurement point in a unit finer than the pixel spacing of the original image.

2. The apparatus according to claim 1, further comprising:
a sampling point travel image generation unit generating an image obtained by moving sampling points by the sampling point travel unit;
an enlarged image generation unit generating an enlarged image by enlarging a sampling point travel image;
an enlarged image display unit displaying an enlarged image; and
a measurement point position specification unit specifying a position of a measurement point on an enlarged image.

3. The apparatus according to claim 1, further comprising
a sampling point travel amount unit specification unit specifying a unit of an amount of travel of sampling points moved by the sampling point travel unit.

4. The apparatus according to claim 2, further comprising
a filter unit performing a filtering process on an enlarged image displayed on the enlarged image display unit.
